SEQUENCE LISTING (9 sequences)

SEQ ID NO: 1 - protein, 39 aa, synthetic construct
  REGION 1..39: note = Synthetic
LSVATALSALSLGAEQRTESIIHRALYYDLISSPDIHGT

SEQ ID NO: 2 - protein, 34 aa, synthetic construct
  REGION 1..34: note = Synthetic
ALSALSLGAEQRTESIIHRALYYDLISSPDIHGT

SEQ ID NO: 3 - protein, 29 aa, synthetic construct
  REGION 1..29: note = Synthetic
SLGAEQRTESIIHRALYYDLISSPDIHGT

SEQ ID NO: 4 - protein, 25 aa, synthetic construct
  REGION 1..25: note = Synthetic
EQRTESIIHRALYYDLISSPDIHGT

SEQ ID NO: 5 - protein, 24 aa, synthetic construct
  REGION 1..24: note = Synthetic
SLGAEQRTESIIHRALYYDLISSP

SEQ ID NO: 6 - protein, 20 aa, synthetic construct
  REGION 1..20: note = Synthetic
SLGAEQRTESIIHRALYYDL

SEQ ID NO: 7 - protein, 18 aa, synthetic construct
  REGION 1..18: note = Synthetic
EQRTESIIHRALYYDLIS

SEQ ID NO: 8 - protein, 29 aa, synthetic construct
  REGION 1..29: note = Synthetic
SLGAEHRTESVIHRALYYDLITNPDIHST

SEQ ID NO: 9 - protein, 20 aa, synthetic construct
  REGION 1..20: note = Synthetic
SLGAEHRTESVIHRALYYDL

(19) United States
(12) Patent Application Publication
(10) Pub. No.: US 2026/0345752 A1
(43) Pub. Date: Nov. 5, 2026

(54) COMPOSITIONS COMPRISING PEDF-DERIVED SHORT PEPTIDES (PDSP) AND USES THEREOF

(71) Applicant: BRIM Biotechnology, Inc., Taipei (TW)

(72) Inventors: Frank Wen-Chi LEE, Orlando, FL (US); Wayne Wei-Cheng LIAW, San Diego, CA (US); Yan-Wei WU, Taipei (TW); Ya-Lang HUANG, Taipei (TW); Chih-Te CHIEN, Taipei (TW)

(73) Assignee: BRIM Biotechnology, Inc., Taipei (TW)

(21) Appl. No.: 19/118,346

(22) PCT Filed: Oct. 3, 2023
(86) PCT No.: PCT/US2023/034383

§ 371 Date: Apr. 3, 2025

Related U.S. Application Data
(60) Provisional application No. 63/412,869, filed on Oct. 3, 2022.

(51) Int. Cl. A61K 38/57; A61K 9/08; A61K 47/22; A61K 47/26; A61K 47/32

(52) U.S. Cl. CPC A61K 38/57; A61K 9/08; A61K 47/22; A61K 47/26; A61K 47/32

(57) ABSTRACT

An aqueous formulation includes a PEDF-derived short peptide (PDSP); a blend of PVP/PVA in different ratios around 8/2-2/8; and a tonicity agent, which is preferably non-ionic. The PDSP is preferably 14-39 amino acids long and has the sequence of one of SEQ ID NO: 1, 2, 3, 5, 6, 8, or 9. The non-ionic tonicity agent is glycerin, sucrose, mannitol, or sorbitol. A concentration of the PDSP is 0.01%-1% w/v.

### BACKGROUND OF INVENTION

### Field of Invention

This invention relates to compositions of PEDF-derived short peptides, particularly to formulations of such peptides and uses thereof.

### Background

Human Pigment Epithelium-derived Factor (PEDF) is a secreted protein of 418 amino acids, with a molecular weight of about 50 kDa. PEDF is a multifunctional protein with many biological functions (see U.S. Patent Application Publication No. 2010/0047212). Different peptide regions of the human PEDF are found to be responsible for different functions. For example, a 34-mer fragment (residues 44-77 of PEDF) has been identified to have anti-angiogenic activity, while a 44-mer fragment (residues 78-121 of PEDF) has been identified to have neurotrophic properties.

Human PEDF-derived short peptides (PDSP) have been found to be promising therapeutics for treating or preventing various diseases or disorders. For example, PDSP are found to be effective in promoting muscle regeneration or arteriogenesis (U.S. Pat. No. 9,884,012), treating alopecia and/or hair depigmentation (U.S. Pat. No. 9,938,328), treating osteoarthritis (U.S. Pat. No. 9,777,048), preventing or ameliorating skin aging (U.S. Pat. No. 9,815,878), treating liver cirrhosis (U.S. Pat. No. 8,507,446), or treating various eye diseases or conditions (e.g., retinal degeneration, Meibomian glad disease, dry eye). Corresponding mouse PEDF-derived short peptides (moPDSP) are also found to have the same therapeutic effects. However, preparations of these peptides were found to lack long-term stabilities. Therefore, there is a need for better formulations for this promising biopharmaceutical product.

### SUMMARY OF THE INVENTION

Embodiments of the invention relate to formulations for a PEDF-derived short peptide (PDSP), including SEQ ID NO: 1 (39-mer), SEQ ID NO: 2 (34-mer), SEQ ID NO: 3 (29-mer), SEQ ID NO: 5 (24-mer), SEQ ID NO: 6 (20-mer), SEQ ID NO: 8 (mo29-mer), and SEQ ID NO: 9 (mo20-mer), wherein mo29-mer and mo20-mer are the mouse PDSPs corresponding to the human 29-mer and 20-mer, respectively. Our recent studies found that the core peptide that retains the biological activities of PDSP lies within the first 14 residues in SEQ ID NO: 3. While embodiments of the invention may use PDSP of any length, preferred embodiments of the invention may contain a PDSP of 14-39 amino acids long.

One aspect of the invention relates to an aqueous formulation that includes a PDSP; polyvinylpyrrolidone (PVP); polyvinyl alcohol (PVA); and a tonicity agent. The tonicity agent is preferably a non-ionic tonicity agent. The non-ionic tonicity agent may be any tonicity agent suitable for pharmaceutical formulations, such as glycerin, sucrose, mannitol, or sorbitol. The PDSP may be 14-39 amino acids long, its N-terminus may be protected as an amide and/or its C-terminus may be an ester or an amide. The PDSP preferably has the sequence of one of SEQ ID NO: 1, 2, 3, 5, 6, 8, or 9. The ratio of PVP/PVA is preferably in a range from 9/1-1/9, such as 8/2, 6/4, 4/6, or 2/8. The aqueous formulation further comprises a buffer, which is preferably a histidine buffer.

According to some embodiments of the invention, the pH value of the aqueous formulations may be around 5-9, preferably around 6-8, more preferably around 6.5-7.5. The non-ionic tonicity agent is preferably sorbitol, which is at a concentration of 10 mM-500 mM, preferably 50-250 mM, more preferably 100-150 mM. A concentration of the PDSP may be 0.01%-10% w/v, preferably 0.01-1% w/v, more preferably around 0.02-0.2% w/v.

Other aspects of the invention would be apparent from the following description and the accompanying drawings.

### BRIEF DESCRIPTION OF THE DRAWINGS

FIG. 1 shows a schematic illustrating a testing protocol for assessing the stabilities of various formulations of PDSP solutions. The combinations among PVP and PVA (hereinafter “PVP/PVA”) were mixed in the selected blend ratios as different PDSP solutions according to the study design. Part I: the selection of different PVP/PVA ratio, the pH values of PDSP solutions were adjusted with 1N HCl or 2N NaOH, the solutions were filtered through 0.2 μm syringe filters, and then each solution was placed in a 20 ml glass bottle with screw cap. The filtered PDSP solutions were stirred at 1,100 RPM at room temperature. Aliquots of 80 μl PDSP solutions were collected at different time points for the determinations of size distribution profiles of small particles in different formulation solutions by dynamic light scattering (DLS). The stirring of PDSP solutions was continued, and the precipitations were further investigated with 300 μl PDSP solution aliquots at 48-hour time points from the start of stirring at 13,000 rpm centrifugation. Part II: Particulate Matter analysis, different PVP/PVA ratio formulations of PDSP solutions were filtered through 0.2 μm syringe filters, and then each solution was placed in a 50 ml glass bottle with screw cap. The filtered PDSP solutions were stirred at 390 RPM at room temperature for 24 hr. Then, the formulation solutions were examined for particulate matters according to U.S. Pharmacopeia (USP) <789> tests. A solution passes the USP <789> test if the average number of particles present in the units tested does not exceed the appropriate values listed in Table 1.

TABLE 1
Count limits for USP <789>

| | | |
|---|---|---|
| Diameter | ≥10 μm | ≥25 μm |
| Number of | 50/mL | 5/mL |
| particles | | |

FIG. 2 shows the results from stability tests of PDSP formulations prepared in different ratios of PVP/PVA mixture formulations (PVP/PVA ratios=10/0, 8/2, 6/4, 4/6, 2/8 and 0/10, respectively) under continuous stirring conditions. These solutions were stirred at 1,100 RPM at room temperature after filtration and centrifugated at 48 hours. The precipitations of different PVP/PVA ratio formulations containing 0.03% PDSP appeared as peaks to the right of the main peaks in these charts.

FIG. 3 shows stability tests of PDSP formulations prepared with different ratios of PVP/PVA mixture formulations under continuous stirring conditions. These solutions, with or without 0.03% PDSP, were continuously stirred at 1,100 RPM at room temperature. These solutions were investigated at 0, 3, 5, 8, 24 and 48 hours by DLS to record the particle size profiles. Finally, the solutions were investigated by centrifugated at 48 hours to assess the particle formation.

### DETAILED DESCRIPTION

Embodiments of the invention relate to formulations of PEDF-derived short peptides (PDSP) with enhanced stabilities. Various human PDSP were found to be promising therapeutics for treating or preventing various diseases or disorders, including muscle regeneration or arteriogenesis, alopecia and/or hair depigmentation, osteoarthritis, skin aging, liver cirrhosis, or eye diseases or conditions. Examples of such PDSP may include those shown in TABLE 2.

TABLE 2
Examples of PEDF derived short peptides (PDSP)

| | | SEQ ID | Human PEDF |
|---|---|---|---|
| Name | Sequence | NO | residues |
| 39mer | LSVATALSALSLGAEQRTESIIHRALYYDLISSPDIHGT | 1 | 82-121 |
| 34mer | ALSALSLGAEQRTESIIHRALYYDLISSPDIHGT | 2 | 88-121 |
| 29mer | SLGAEQRTESIIHRALYYDLISSPDIHGT | 3 | 93-121 |
| 25mer | EQRTESIIHRALYYDLISSPDIHGT | 4 | 97-121 |
| 24mer | SLGAEQRTESIIHRALYYDLISSP | 5 | 93-116 |
| 20mer | SLGAEQRTESIIHRALYYDL | 6 | 93-112 |
| 18mer | EQRTESIIHRALYYDLIS | 7 | 97-114 |
| mo29mer | SLGAEHRTESVIHRALYYDLITNPDIHST | 8 | mouse |
| mo20mer | SLGAEHRTESVIHRALYYDL | 9 | mouse |

In accordance with embodiments of the invention, the PDSP may be SEQ ID NO: 1, 2, 3, 5, 6, 8, or 9. In addition, the N-termini of these peptides may be optionally protected with acylation (e.g., acetyl or propionyl protection), and the C-termini may be optionally protected as amides.

These PDSP have been prepared in citrate buffers and found to be effective for therapeutic purposes in various pre-clinical studies. However, preparations of these short peptides (e.g., BRM421, 29mer PDSP (SEQ ID NO:3) in 10 mM citrate buffer with 0.85% w/v NaCl, pH 6.0) were found to lack long-term stabilities (over several months).

Many factors, including chemical stress (e.g., oxidation, hydrolysis, etc.) and physical stress (e.g., temperature, light, and agitation), can affect the qualities and stabilities of biopharmaceutical products, particularly during long-term storage. To investigate the stabilities of PDSP in different formulations, accelerated stability testing was performed. Specifically, various formulations were tested under stress conditions, particularly under shear stress, to identify optimal formulations.

Polyvinylpyrrolidone (PVP) is a water-soluble, inert, non-toxic, pH-stable, biocompatible polymer that helps to solubilize both hydrophilic and lipophilic drugs. These advantages enable PVP to serve as a versatile excipient in the drug formulation development. Polyvinyl alcohol (PVA) is a water-soluble polymer that is often used to increase viscosities in pharmaceuticals and as a lubricant and protectant in ophthalmic preparations. The use of PVP/PVA combination in ophthalmic formulations was found to have soothing effects. This combination was investigated for its ability to provide long-term stabilities of PDSP formulations. Embodiments of the invention show that the stabilities of PDSP solutions could be enhanced in certain PVP/PVA ratios.

The following describes specific examples to illustrate embodiments of the invention. However, one skilled in the art would appreciate that these specific examples are for illustration only and that other modifications and variations are possible without departing from the scope of the invention. For example, even though the following examples use BRM421 (29mer PDSP; SEQ ID NO:3) for illustrations, other PDSP may be used instead.

### 1. Preparation of Different PVP/PVA Ratio Formulations

The PVP/PVA formulations may be used at any suitable concentrations, e.g., 0.5-10% w/v, preferably 1-5% w/v, more preferably 2-4% w/v. PVP and PVA can be readily sourced from commercial sources (e.g., Sigma-Aldrich). Suitable PVP for use with embodiments of the invention may have a number-average molecular weight in a range of 10-400 kDa, preferably 10-100 kDa, more preferably 20-60 kDa. PVA suitable for use with embodiments of the invention may have a weight-average molecular weight range of 10-100 kDa, preferably 10-50 kDa, more preferably 10-30 kDa.

As an example, the following describes a formulation of 2% w/v PVP/PVA formulations. One of ordinary skilled in the art would appreciate that these examples are for illustration only and other % (e.g., 3%, 4%, etc.) and/or PVP/PVA ratios may be similarly prepared. Solutions of 2% PVP/PVA in different ratios were prepared as follows. First, a 2% PVP (number-average MW about 60,000) and 2% PVA (weight-average MW about 13,000), respectively, solutions were prepared in distilled water. Then, they were physically blended in the ratios of 10/0, 8/2, 6/4, 4/6, 2/8 and 0/10 for the studies. For example, for a 6/4 PVP/PVA ratio 8 mL solution, the following material volumes were prepared:
8 mL, 2% PVP/PVA ratio=6/4:4.8 mL, 2% PVP+3.2 mL, 2% PVA

Various tonicity adjusting agents have been used in ophthalmic formulations, such as citrate (MW 192.12), NaCl (MW 58.44), sorbitol (MW 182.17), and histidine (MW 155.15). The above-mentioned tonicity adjusting agents may be added to the PVP/PVA solutions to prepare a series of ophthalmic formulations for evaluation of their capacities to ensure long-term stabilities. The following are some examples:
1. Different 2% PVP/PVA ratio solutions with Citrate Buffer (55 mM Citrate with 45 mM NaCl, pH7.5);
2. Different 2% PVP/PVA ratio solution with Citrate Buffer (55 mM Citrate with 137 mM Sorbitol, pH7.5); and
3. Different 2% PVP/PVA ratio solution with Histidine Buffer (125 mM Histidine with 137 mM Sorbitol, pH7.6).

### 2. Preparation of PDSP in Different Formulations

The main PDSPs used in these examples include BRM421 (29mer) (SEQ ID NO:3, MW 3243.6; peptide content: 88.6%), 39mer (SEQ ID NO:1; MW 4211.71; peptide content: 96.5%), and 20mer (SEQ ID NO:6, MW 2376.63; peptide content: 95.2%). PDSP may be used at any suitable concentrations (e.g., 0.01%-5% w/v, preferably 0.01%-1% w/v, and more preferably 0.01%-0.1% w/v). In the following examples, the PDSP concentration is 0.03% w/v (about 0.1 mM). For example, the calculation formula for BRM421 (29mer) in a 30 mL solution is as follow:
0.03% BRM421=0.03 g/100 ml=0.0003 g/ml (about 0.1 mM)
0.0003 g÷88.6% peptide content=0.0003386 g
0.0003386×30=0.010158 g
e.g., 30 ml 2% PVP/PVA ratio (8/2) solution with citrate buffer+0.010158 g BRM421=30 ml, 0.03% BRM421

The pH values of formulations were measured after PDSP completely dissolved in the solutions and then adjusted to 7.5 or 7.6 according to the study designs. Before use in the study, PDSP formulation solutions were filtered through a syringe filter (e.g., a 0.2 μm filter).

3. Evaluation of PDSP Prepared in Different Formulations for their Abilities to Resist Shearing Forces

We noticed that earlier formulations of PDSP in citrate buffers were not stable during long-term storage. To test the effects of different PVP/PVA formulations on the stabilities, the various PDSP formulations were subject to stress conditions (e.g., shear stress) to accelerate the changes.

For study Part I: selection of different PVP/PVA ratios, 29mer PDSP solutions prepared in different buffers and excipients (as shown in Table 3) were subject to stirring at 1,100 RPM at room temperature. Aliquots of 80 μl PDSP solutions each were collected into 1.5 ml Eppendorf tubes at different time points to determine the size distribution profiles of small particles in different formulation solutions using dynamic light scattering (DLS). The stirring of PDSP solutions was continued, and the precipitations were further investigated with 300 μl PDSP solutions at 48-hour time points from the start of stirring by centrifugation at 13,000 rpm.

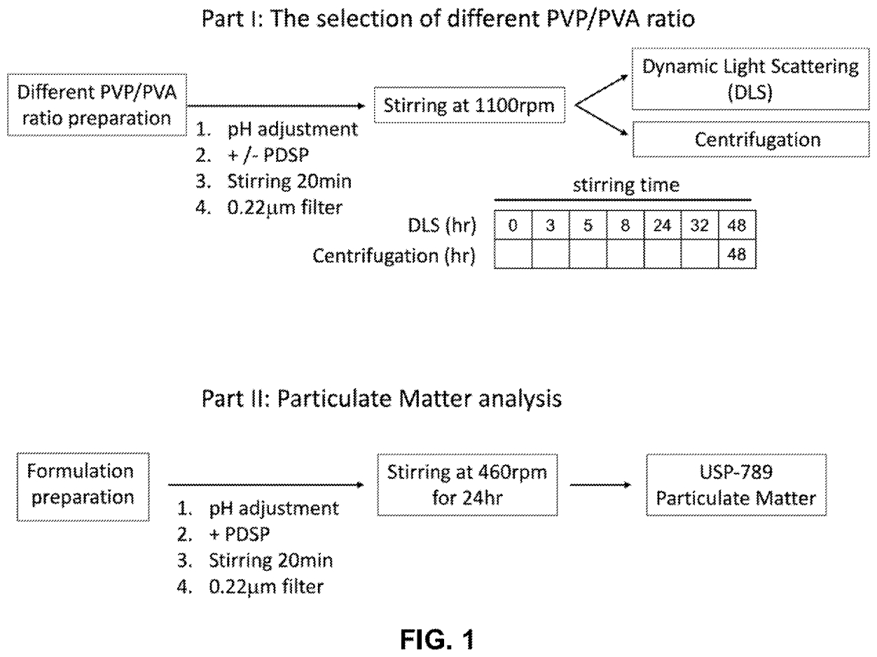

For Part II: Particulate Matter analysis, different PVP/PVA ratio formulations of PDSP solutions were stirred at 390 RPM at room temperature for 24 hr. Then, the formulation solutions were evaluated according to USP <789> protocols. The formulation passes the USP <789> test if the average number of particles present in the unit does not exceed the appropriate values. The experimental procedures are illustrated in FIG. 1.

TABLE 3
List of the formulations tested in this study.

| | pH |
|---|---|
| | value |
| Formulation (Study Part I) | |
| 2% PVP/PVA (10/0), 55 mM citrate, 137 mM sorbitol | 7.5 |
| 2% PVP/PVA (8/2), 55 mM citrate, 137 mM sorbitol | 7.5 |
| 2% PVP/PVA (6/4), 55 mM citrate, 137 mM sorbitol | 7.5 |
| 2% PVP/PVA (4/6), 55 mM citrate, 137 mM sorbitol | 7.5 |
| 2% PVP/PVA (2/8), 55 mM citrate, 137 mM sorbitol | 7.5 |
| 2% PVP/PVA (0/10), 55 mM citrate, 137 mM sorbitol | 7.5 |
| Formulation (Study Part II) | |
| 2% PVP/PVA (8/2), 55 mM citrate, 45 mM NaCl | 7.6 |
| 2% PVP/PVA (8/2), 55 mM citrate, 137 mM sorbitol | 7.6 |
| 2% PVP/PVA (8/2), 125 mM histidine, 137 mM sorbitol | 7.6 |
| 2% PVP/PVA (6/4), 125 mM histidine, 137 mM sorbitol | 7.6 |
| 2% PVP/PVA (4/6), 125 mM histidine, 137 mM sorbitol | 7.6 |
| 2% PVP/PVA (2/8), 125 mM histidine, 137 mM sorbitol | 7.6 |

### Results

1. The Ability to Resist Shearing Force for 29mer PDSP Prepared in Different PVP/PVA Ratio Solutions (10/0, 8/2, 6/4, 4/6, 2/8, and 0/10, Respectively) with 55 mM Citrate with 137 mM Sorbitol, pH 7.5

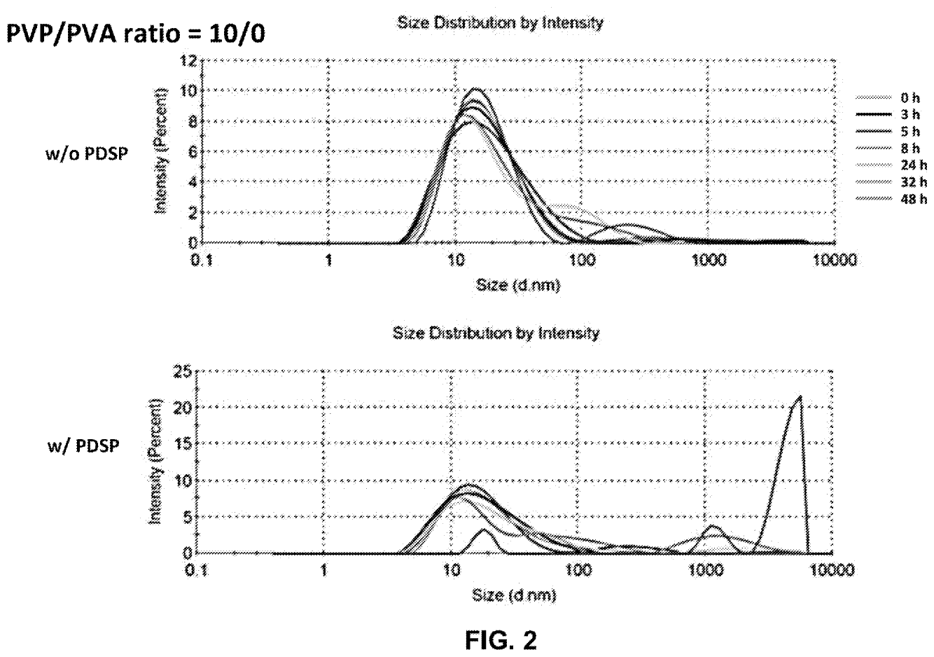

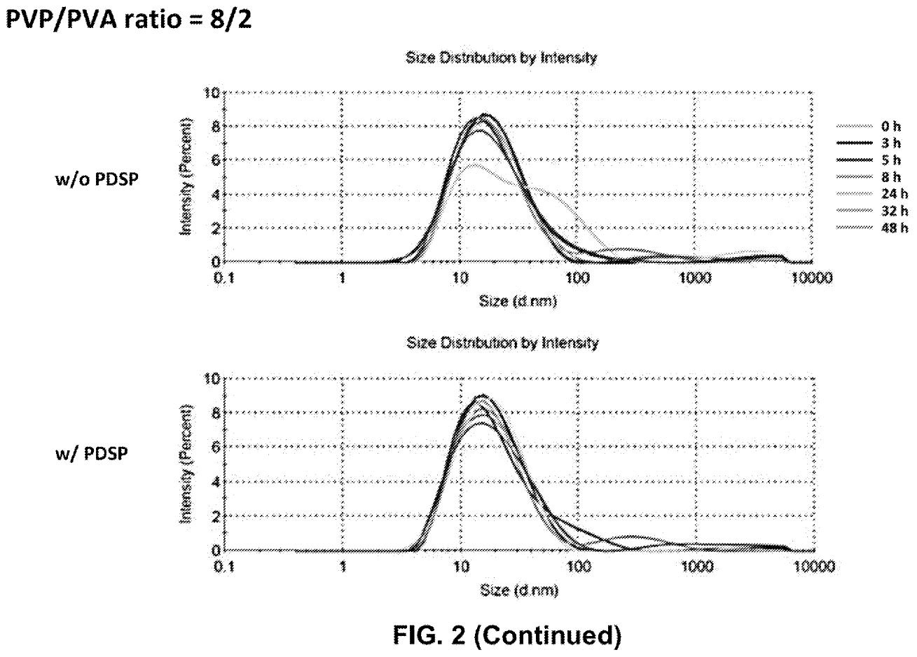

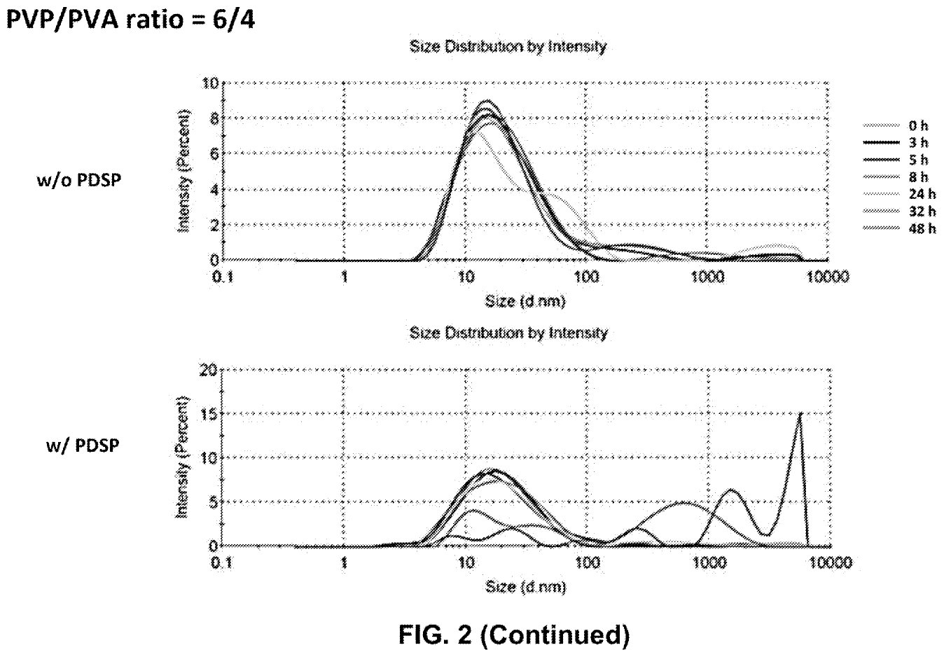

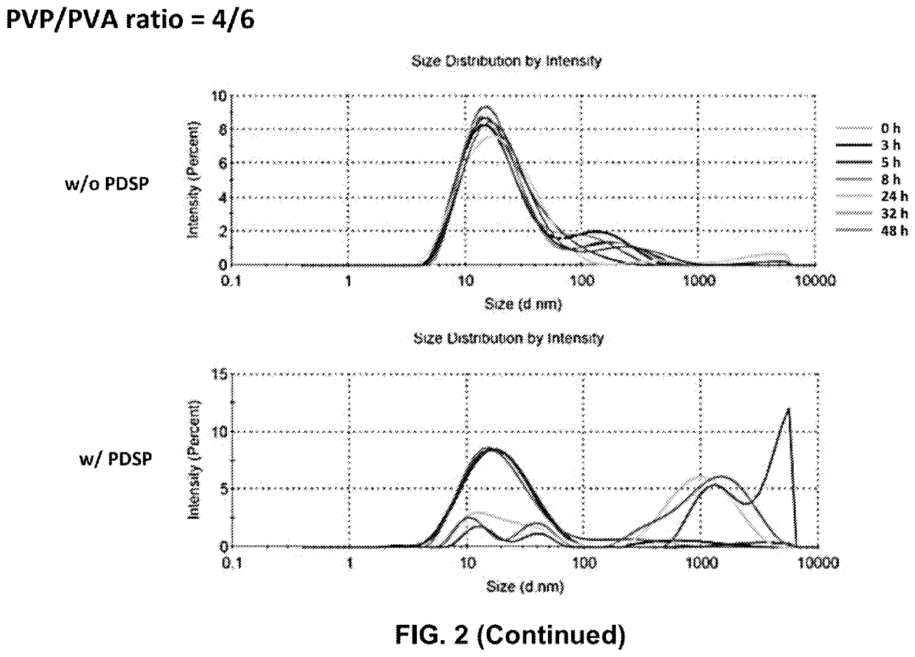

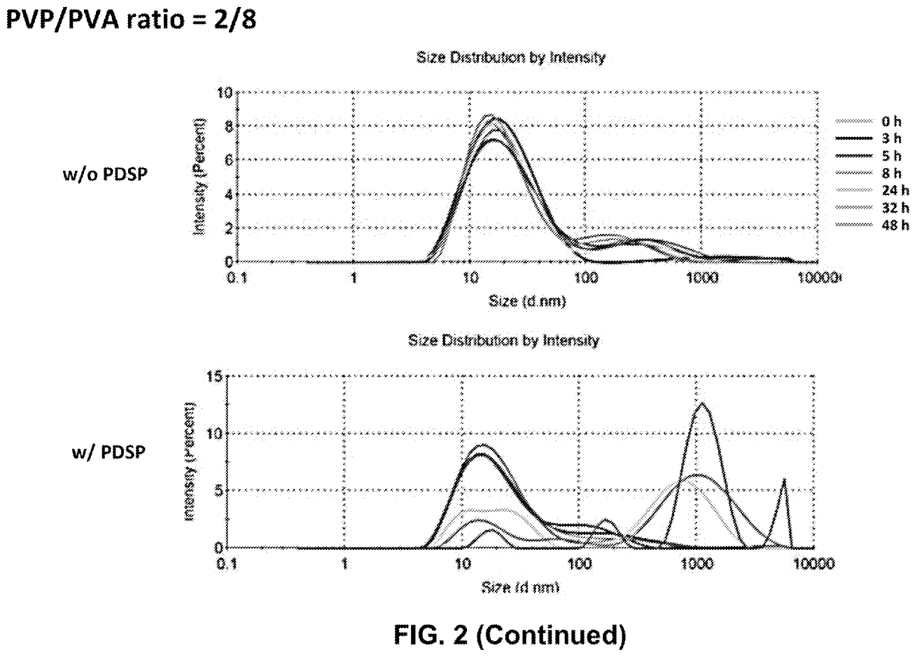

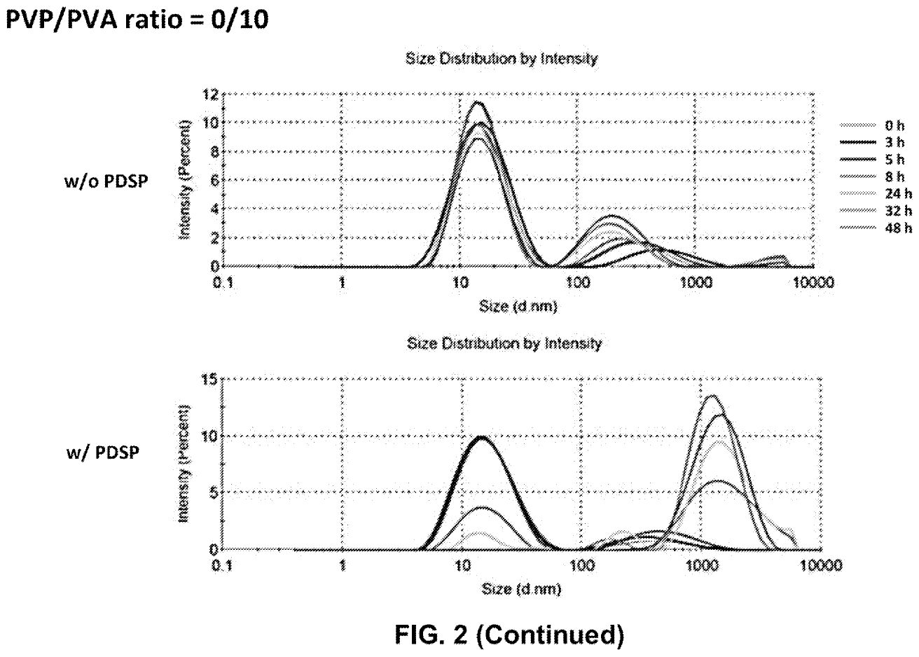

The stabilities of PDSP prepared in different PVP/PVA ratio formulations under stirring conditions were investigated, using DLS and centrifugation analysis as the screening tools, to elucidate the abilities of these formulations to resist shearing force. The DLS results as shown in FIG. 2 indicate that the major particle size dispersions were consistently between 5 nm and 100 nm at different time points for the PDSP formulation with a PVP/PVA ratio=8/2, suggesting that the PVP/PVA ratio=8/2 formulation is the best among the test samples for providing protection against shearing force effects. In contrast, all other PVP/PVA ratios are less effective in protecting the PDSP formulations from shearing forces, as evidenced by substantial precipitate/aggregate formations.

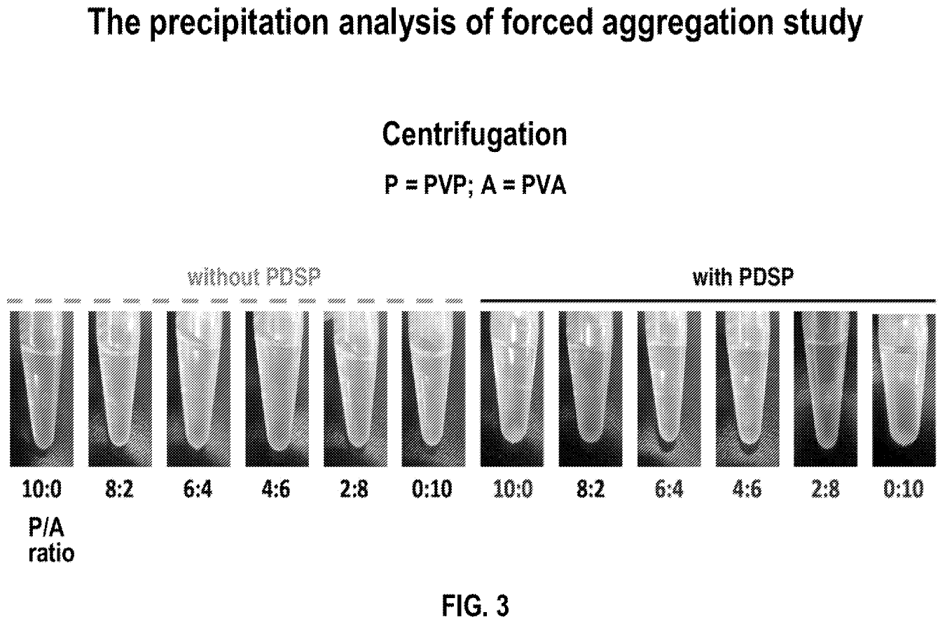

In aqueous solutions, precipitation results from a process of transforming dissolved PDSP into an insoluble solid from in the formulation solution under stress conditions. At 48 hours after the start of stirring, precipitation analysis, based on high-speed centrifugation pellet formation, shows that no precipitate was observed for the PVP/PVA ratio=8/2 formulation. In contrast, other formulations show appreciable precipitations (FIG. 3). These data indicate that 2% w/v PVP/PVA formulations with a ratio of around 8/2 are more suitable for PDSP stabilities. However, as will be described in latter sections, different ratios of PVP/PVA may be preferred at different concentrations or with different buffers and/or tonicity agents.

2. Particulate Matter Analysis of 29mer PDSP Prepared in PVP/PVA Ratio=8/2 Based Formulations with Different Excipients Under Stirring Conditions

Ophthalmic solutions should be free from particulate matters that can be observed on visual inspection. USP <789> describes tests for enumerating particles with specific size ranges. Various PDSP formulations with different excipients and a PVP/PVA ratio of 8/2 were assessed for particular matters according to USP <789> tests. The PDSP formulation with PVP/PVA ratio=8/2, 55 mM citrate, 137 mM sorbitol, pH 7.5 was the initial formulation. However, this formulation was not good for PDSP formulation development due to a large volume of pH adjustment to pH 7.5. Therefore, other PDSP formulations with PVP/PVA ratio=8/2 and other tonicity adjusting agents were investigated.

In accordance with USP <789> Particulate Matter in ophthalmic solutions tests, various PVP/PVA ratio=8/2 based PDSP formulations with other tonicity agents were evaluated under stirring conditions. The results are shown in Table 4. Because the results of formulation with 45 mM NaCl were not reproducible, the formulation with PVP/PVA ratio=8/2, 125 mM histidine, 137 mM sorbitol, pH7.6 was chosen as the best for PDSP ophthalmic solutions.

TABLE 4
The Particulate Matter analysis of 29mer PDPS prepared
in different formulations under stirring conditions

| | | USP < 789 > ¹ | |
|---|---|---|---|
| | | ≥10 | ≥25 |
| | Analysis | μM | μM |
| 2% PVP/PVA Formulation/pH | times | (50/ml) | (5/ml) |
| PVP/PVA (8/2), 55 mM citrate, | first | 23.8 | 1.4 |
| 137 mM sorbitol/pH 7.5 | | | |
| PVP/PVA (8/2), 55 mM citrate, | second | 34.2 | 0.8 |
| 137 mM sorbitol/pH 7.5 | | | |
| PVP/PVA (8/2), 55 mM citrate, | first | 7.0 | 0.5 |
| 45 mM NaCl/pH 7.5 | | | |
| PVP/PVA (8/2), 55 mM citrate, | second | 186.0 ² | 3.4 |
| 45 mM NaCl/pH 7.5 | | | |
| PVP/PVA (8/2), 125 mM histidine, | first | 9.1 | 1.3 |
| 137 mM sorbitol/pH 7.6 | | | |
| PVP/PVA (8/2), 125 mM histidine, | second | 4.2 | 0.4 |
| 137 mM sorbitol/pH 7.6 | | | |

¹ these 3 sets of study had been conducted twice to confirm the results and the same results from two formulations were obtained.
² out of specification

### 3. The Ability to Resist Shearing Stress for Different PEDF Derived Short Peptides Prepared in Different 2% and 4% PVP/PVA Ratio Solutions, Respectively

The above experiments regarding PVP/PVA based formulation studies were performed with the 29mer synthetic peptide, SEQ ID NO:3. In order to investigate whether other PEDF derived short peptides are also protected against stirring force by PVP/PVA based formulations, the other PDSP peptides, e.g., 39mer (SEQ ID NO:1) and 20mer (SEQ ID NO: 6), were prepared in different PVP/PVA ratio formulations, as described in Table 3. Study Part II, using these other PDSP, was performed in the same shearing force challenge. The results for the 39mer and 20mer are shown in Table 5. These results together suggest that, in addition to PVP/PVA ratio=8/2, other PVP/PVA ratio formulations (PVP/PVA based solution with 125 mM histidine and 137 mM sorbitol, pH7.6) can provide protection against shearing stress, even for longer peptides (e.g., the 39mer), and that PVP/PVA formulations with different PDSP (e.g., SEQ ID NO: 1, 2, 3, 5, 6, 8, and 9) also have enhanced stabilities.

As compared to PDSP in citrate buffers, the PVP/PVA formulations are markedly more stable under the same stress conditions, indicating the superiority of the PVP/PVA formulations.

TABLE 5
The particulate matter analysis of different PDSP prepared in
different PVP/PVA ratio formulations under stirring conditions

| | | USP < 789 > | |
|---|---|---|---|
| | | ≥10 | ≥25 |
| | PDSP | μM | μM |
| 2% PVP/PVA Formulations/pH | length | (50/ml) | (5/ml) |
| PVP/PVA (8/2), 125 mM histidine, | 39mer | 33.3 | 2.1 |
| 137 mM sorbitol, pH 7.6 | | | |
| PVP/PVA (6/4), 125 mM histidine, | 39mer | 77.2* | 3.9 |
| 137 mM sorbitol, pH 7.6 | | | |
| PVP/PVA (4/6), 125 mM histidine, | 39mer | 45.2 | 0.9 |
| 137 mM sorbitol, pH 7.6 | | | |
| PVP/PVA (2/8), 125 mM histidine, | 39mer | 21.1 | 1.1 |
| 137 mM sorbitol, pH 7.6 | | | |
| PVP/PVA (8/2), 125 mM histidine, | 29mer | 7.4 | 0.4 |
| 137 mM sorbitol, pH 7.6 | | | |
| PVP/PVA (6/4), 125 mM histidine, | 29mer | 9.9 | 0.4 |
| 137 mM sorbitol, pH 7.6 | | | |
| PVP/PVA (4/6), 125 mM histidine, | 29mer | 17.8 | 0.4 |
| 137 mM sorbitol, pH 7.6 | | | |
| PVP/PVA (2/8), 125 mM histidine, | 29mer | 28.6 | 1.8 |
| 137 mM sorbitol, pH 7.6 | | | |
| PVP/PVA (8/2), 125 mM histidine, | 20mer | 16.3 | 0.3 |
| 137 mM sorbitol, pH 7.6 | | | |
| PVP/PVA (6/4), 125 mM histidine, | 20mer | 53.6* | 3.9 |
| 137 mM sorbitol, pH 7.6 | | | |
| PVP/PVA (4/6), 125 mM histidine, | 20mer | 2.8 | 0.2 |
| 137 mM sorbitol, pH 7.6 | | | |
| PVP/PVA (2/8), 125 mM histidine, | 20mer | 31.1 | 0.6 |
| 137 mM sorbitol, pH 7.6 | | | |
| 10 mM citrate, 0.85% NaCl/pH 6.0 | 29mer | 16,610* | 5,605* |
| (as negative control) | | | |

*out of specification

The above examples clearly show that new formulations of PDSP in different PVP/PVA-based solutions with histidine or other tonicity agents can afford dramatically improved stabilities, as compared to the original formulations in citrate-based buffers.

Further experiments were performed to examine the effects of different PVP/PVA concentrations on the stabilities of PDSP formations. As shown in Table 6 below, with 4% PVP/PVA, ratios of 4:6 and 2:8 PVP/PVA produced better results. These results indicate that different concentrations and different ratios of PVP/PVA may be selected to suit different applications in accordance with embodiments of the invention.

TABLE 6
Different ratios of 4% PVP/PVA formulations
PVP/PVA (8:2; 6:4; 4:6; 2:8), 125 mM Histidine,
133 mM Sorbitol, 29mer PDSP (0.1 mM)

| | | PVP/ | PVP/ | PVP/ | PVP/ |
|---|---|---|---|---|---|
| | 350 mM | PVA | PVA | PVA | PVA |
| | Nicotinamide# | (8:2) | (6:4) | (4:6) | (2:8) |
| pH | 6.99 | 7.45 | 7.54 | 7.47 | 7.50 |
| ≥10 μm | 0.8 | 65.80* | 80.65* | 35.35 | 45.55 |
| ≥25 μm | 0.05 | 1.85 | 4.5 | 1.40 | 1.40 |
| Osmolarity | 289 | 302 | 308 | 317 | 323 |

#PDSP 0.1 mM in 350 mM nicotinamide, 20 mM histidine (without PVP/PVA) for comparison
*out of specification

Further experiments were performed to examine the effects of different concentrations of PDSP (e.g., 29mer) in PVP/PVA (4:6, 125 mM histidine, 130 mM sorbitol) on the stabilities of PDSP formulations. As shown in Table 7 below, in PVP/PVA (4:6, 125 mM histidine, 130 mM sorbitol), PDSP at 0.1-0.4 mM concentrations all have satisfactory stabilities (meet USP <789> requirements) after 24 hr forced aggregation (390 rpm stirring) at room temperature. These results indicate that PVP/PVA formulations of the invention can improve the stabilities of PDSP solutions at various concentrations for therapeutic applications.

TABLE 7
Different concentrations of PDSP in 0.2 mM PVP/0.66
mM PVA (4:6), 125 mM Histidine, 130 mM Sorbitol

| | 350 mM | | | | | | | |
|---|---|---|---|---|---|---|---|---|
| | Nico# | PDSP | PDSP | PDSP | PDSP | PDSP | PDSP | PDSP |
| | (0.1 mM) | (0.1 mM) | (0.1 mM) | (0.2 mM) | (0.2 mM) | (0.3 mM) | (0.3 mM) | (0.4 mM) |
| pH | 7.05 | 7.51 | 7.49 | 7.43 | 7.44 | 7.45 | 7.43 | 7.43 |
| ≥2 μm | 36.55 | 56.95 | 213.7 | 286.3 | 192.35 | 509.10 | 228.65 | 308.05 |
| ≥5 μm | 7.4 | 9.35 | 44.75 | 54.65 | 47.65 | 119.70 | 48.40 | 76.80 |
| ≥10 μm | 1.35 | 2.50 | 8.95 | 7.40 | 12.60 | 25.00 | 9.30 | 20.50 |
| ≥15 μm | 0.55 | 0.4 | 2.25 | 0.95 | 3.00 | 2.70 | 1.90 | 1.85 |
| ≥25 μm | 0.05 | 0.05 | 0.25 | 0.05 | 0.40 | 0.15 | 0.60 | 0.25 |
| Osmol. | 291 | 284 | 284 | 288 | 285 | 286 | 285 | 285 |

#PDSP 0.1 mM in 350 mM nicotinamide, 20 mM histidine (without PVP/PVA) for comparison;
PVP K30: Sigma [catalog 81420] average MW = 40,000;
PVA Sigma [catalog 348406] average MW = 13,000-23,000, 98% hydrolyzed.

The above results show that PVP/PVA at various ratios (e.g., 2/8-8/2) and various concentrations (e.g., 1%, 2%, 3%, 4%, 5% w/v, etc.) can be used to promote the stabilities of PDSP formulations in accordance with embodiments of the invention. These PVP/PVA formulations can be used with various concentrations of PDSP (e.g., 0.01 mM-10 mM, preferably 0.1-1 mM, more preferably 0.1-0.5 mM). As compared to the citrate formulations of PDSP, the PVP/PVA formations of PDSP are dramatically more stable (see Table 5).

Embodiments of the invention have been illustrated with a limited number of examples. One skilled in the art would appreciate that these examples are for illustration only and are not meant to limit the scope of the invention because other modifications and variations are possible without departing from the scope of the invention. Accordingly, the scope of the invention should be limited by the attached claims.

1. An aqueous formulation, comprising:
a pigmented epithelium-derived factor (PEDF)-derived short peptide (PDSP);
a blend of polyvinylpyrrolidone (PVP) and polyvinyl alcohol (PVA), and a tonicity agent.

2. The aqueous formulation of claim 1, wherein the PDSP is 14-39 amino acids long.

3. The aqueous formulation of claim 1, wherein the PDSP has the sequence of SEQ ID NO: 1, 2, 3, 5, 6, 8, or 9.

4. The aqueous formulation of claim 1, wherein the blend of PVP and PVA has a PVP/PVA ratio of 8/2, 6/4, 4/6, or 2/8.

5. The aqueous formulation of claim 1, wherein a pH value of the aqueous formulation is 6.0-8.0.

6. The aqueous formulation of claim 1, wherein the tonicity agent is a non-ionic tonicity agent.

7. The aqueous formulation of claim 6, wherein the non-ionic tonicity agent is glycerin, sucrose, mannitol, or sorbitol.

8. The aqueous formulation of claim 6, the non-ionic tonicity agent is sorbitol.

9. The aqueous formulation of claim 1, further comprising histidine.

10. The aqueous formulation of claim 1, wherein a concentration of the PDSP is 0.01%-1% w/v.
